(19) United States
(12) Patent Application Publication
(10) Pub. No.: US 2016/0340437 A1
(43) Pub. Date: Nov. 24, 2016

(54) Methods for Managing Sepsis

(71) Applicant: Emory University, Atlanta, GA (US)

(72) Inventors: Mandy L. Ford, Atlanta, GA (US); Craig Coopersmith, Atlanta, GA (US)

(21) Appl. No.: 15/159,297

(22) Filed: May 19, 2016

Related U.S. Application Data
(60) Provisional application No. 62/163,429, filed on May 19, 2015.

(51) Int. Cl. C07K 16/28

(52) U.S. Cl. CPC C07K 16/2896; C07K 16/2803; C07K 2317/565; A61K 2039/505

(57) ABSTRACT

This disclosure relates to methods of treating or preventing sepsis, severe sepsis, or septic shock comprising administering an effective amount of a 2B4 antibody, a CD48 variant, a CD48 antibody or specific binding agent thereof to a subject in need thereof. In certain embodiments, the subject is in need thereof because the subject is diagnosed with, exhibiting symptoms of, or at risk for sepsis.

### CROSS REFERENCE TO RELATED APPLICATIONS

This application claims the benefit of priority to U.S. Provisional Application No. 62/163,429 filed May 19, 2015, hereby incorporated by reference in its entirety.

### STATEMENT REGARDING FEDERALLY SPONSORED RESEARCH OR DEVELOPMENT

This invention was made with government support under RO1AI104699 and R01GM072808 awarded by the NIH. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 14111US_ST25.txt. The text file is 12 KB, was created on May 19, 2016, and is being submitted electronically via EFS-web.

### BACKGROUND

Sepsis typically result from an uncontrolled bacterial infection and is the leading cause of death among critically ill patients. Outside of antibiotics, treatment of sepsis is non-specific, aimed at early cardiopulmonary resuscitation to minimize the adverse systemic effects of the infection. Anti-inflammatory therapies have been tested to address the inflammatory aspects. However, treatment with immunomodulatory agents have not been successful in clinical trials. Recombinant human activated protein C (Drotrecogin alfa) was approved for the treatment of severe sepsis on the basis of a prospect evaluation, but later found not to be beneficial when administered to a population of patients for which it was an approved treatment. Ranieri et al., N Engl J Med, 2012, 366:2055-2064. Therapeutic options are limited once antibiotics and supportive therapy fail. Thus, there is a need to identify improved methods for managing sepsis.

Boomer et al. report immunosuppression in patients who die of sepsis and multiple organ failure. JAMA, 2011, 306(23):2594-605. Hutchins et al. report that cytokines such as IL-7, IL-15, GM-CSF as well as co-inhibitory molecule blockade, such as anti-PD-1 and anti-BTLA, may be useful in alleviating the clinical morbidity associated with sustained sepsis. Trends Mol Med. 2014, 20(4): 224-233.

References cited herein are not an admission of prior art.

### SUMMARY

This disclosure relates to methods of treating or preventing sepsis, severe sepsis, or septic shock comprising administering an effective amount of a 2B4 antibody, a CD48 variant, a CD48 antibody or specific binding agent thereof to a subject in need thereof. In certain embodiments, the subject is in need thereof because the subject is diagnosed with, exhibiting symptoms of, or at risk for sepsis.

In certain embodiments, the specific binding agent specifically binds the extracellular domain of human 2B4 or CD48. In certain embodiments, the specific binding agent is a monoclonal antibody. In certain embodiments, the specific binding agent or antibody is a humanized antibody, chimera, or fragment thereof.

In certain embodiments, the 2B4 antibody, CD48 variant, a CD48 antibody or specific binding agent is administered in combination with an antibiotic, antiviral, other anti-infections agent, or active agent.

### BRIEF DESCRIPTION OF THE FIGURES

FIG. 1A shows data indicating 2B4 expression was increased on T cells following cecal ligation and puncture (CLP), a mouse model of polymicrobial sepsis. WT mice underwent CLP or sham surgery and were sacrificed at various time points as indicated. Splenocytes were harvested and stained for NK cells. Isotype control was used to gate on the 2B4+ population. For CD4+ and CD8+ T cells, CD3+NK1.1—were gated to rule out NKT cells.

FIG. 1B shows data when splenocytes were harvested and stained for CD4+ T cells.

FIG. 1C shows data when plenocytes were harvested and stained for CD8+ T cells.

FIG. 2A shows data indicating 2B4 expression is associated with decreased TNF and IL-2 production in CD4+ T cells. WT mice underwent CLP or sham surgery and were sacrificed at 24 hours post-CLP. Splenocytes were stimulated in PMA and Ionomycin for 4 hours. IFN-gamma was stained to determine CD4 T cell functionality.

FIG. 2B shows data when TNFalpha was stained to determine CD4 T cell functionality.

FIG. 2C shows data when IL-2 was stained to determine CD4 T cell functionality.

FIG. 3A shows data indicating 2B4-deficient mice were protected from CLP. CLP was performed on WT and 2B4-deficient mice and animals were monitored for 7 days survival curve.

FIG. 3B shows data when the blood and peritoneal fluid was collected for bacteria culture at 24 hours post surgery, and the colony-forming units (CFUs) was determined after 24 hours culture.

FIG. 3C shows data when splenocytes were harvested at 24 hours post surgery and the lymphocytes count were assessed by flow cytomyetry.

FIG. 4A shows data indicating CD4 T cells in 2B4-deficient mice exhibited increased effector functions during sepsis. WT and 2B4-deficient mice were sacrificed at 24 hours post CLP, CD4 T cells in splenocytes were harvested and stained for different surface markers. The percentage of effector and memory markers: CD44, CD69 and KLRG-1 on CD4 T cells in WT mice and 2B4-deficient mice were shown.

FIG. 4B shows data on cytokine receptor expression, CD127 MFI and the percentage of CD122 and CD25 were shown.

FIG. 4C shows data on co-stimulatory receptors: CD27 MFI, CD28 MFI and ICOS+ percentage were shown.

FIG. 4D shows data on co-inhibitory receptors: PD-1, BTLA and LAG-3 percentage in CD4 T cells were shown.

FIG. 4E shows data for cytokine staining, splenocytes were stimulated in PMA and lonomycin for 4 hours.

FIG. 5A shows experiments that indicated the loss of 2B4 specifically on CD4 T cells provides the survival benefit during sepsis. The generation of CD4-specific 2B4−/− chimeric mice. Splenocytes were harvested at 24 hours post surgery and stimulated in PMA and Ionomycin for IFN-gamma, TNF-alpha and IL-2 staining.

FIG. 5B shows data after 10 weeks of reconstitution, the animals were bled for confirmation of phenotype. CD4 and CD8 T cells were gated on CD45.1+CD3+. The control chimera and CD4-specific 2B4−/− chimeric were subjected to CLP and monitored for 7 days survival.

FIG. 5C shows data when the blood and peritoneal fluid was collected and bacteria cultures were determined at 24 hours post surgery.

FIG. 5D shows data when the functionality of CD4 T cells in chimeric mice were assessed by intracellular cytokine staining. IFN-g, TNF and IL-2 were stained to determine CD4 T cell functionality.

FIG. 6 shows data indicating that a pharmacologic blockade of 2B4 results in decreased mortality following CLP. WT mice underwent CLP and were treated with either PBS or anti-2B4 mAb (500 ug/dose on days 0 and 2). Mice were monitored for survival. p=0.053.

FIG. 7 shows data indicating PBMC isolated from human septic patients increased upregulation of 2B4 in both CD4+ ad CD8+ T cell populations. Septic patients were enrolled in an IRB-approved protocol and PBMC were taken at 24 hours post diagnosis of sepsis or septic shock, and analyzed by flow cytometry.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In the claims appended hereto, the term “a” or “an” is intended to mean “one or more,” and the term “comprise” and variations thereof such as “comprises” and “comprising,” when preceding the recitation of a step or an element, are intended to mean that the addition of further steps or elements is optional and not excluded.

The terms “treatment” or “treating” include any desirable effect on the symptoms or pathology of a disease or condition, and may include even minimal reductions in one or more measurable markers of the disease or condition being treated. “Treatment” does not necessarily indicate complete eradication or cure of the disease or condition, or associated symptoms thereof. The subject receiving this treatment is any animal in need, including primates, in particular humans, and other mammals such as equines, cattle, swine and sheep; and poultry and pets in general.

The terms “protein” and “polypeptide” refer to compounds comprising amino acids joined via peptide bonds and are used interchangeably. Amino acids may be naturally or non-naturally occurring.

A “variant” refers to a chemically similar sequence because of amino acid changes or chemical derivative thereof. In certain embodiments, a variant contains one, two, or more amino acid deletions or substitutions. In certain embodiments, the substitutions are conserved substitutions. In certain embodiments, a variant contains one, two, or ten or more an amino acid additions. The variant may be substituted with one or more chemical substituents.

One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Certain variants have less than 10%, and preferably less than 5%, and still more preferably less than 2% changes (whether substitutions, deletions, and so on).

As used herein, the terms “prevent” and “preventing” include the prevention of the recurrence, spread or onset. It is not intended that the present disclosure be limited to complete prevention. In some embodiments, the onset is delayed, or the severity of the recurrence, spread or onset is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “2B4” refers to refers to the naturally occurring molecule in humans identified as having (SEQ ID NO: 17) MLGQVVTLIL LLLLKVYQGK GCQGSADHVV SISGVPLQLQ PNSIQTKVDS IAWKKLLPSQNGFHHILKWE NGSLPSNTSN DRFSFIVKNL SLLIKAAQQQ DSGLYCLEVT SISGKVQTATFQVFVFESLL PDKVEKPRLQ GQGKILDRGR CQVALSCLVS RDGNVSYAWY RGSKLIQTAGNLTYLDEEVD INGTHTYTCN VSNPVSWESH TLNLTQDCQN AHQEFRFWPF LVIIVILSAL FLGTLACFCV WRRKRKEKQS ETSPKEFLTI YEDVKDLKTR RNHEQEQTFP GGGSTIYSMIQSQSSAPTSQ EPAYTLYSLI QPSRKSGSRK RNHSPSFNST IYEVIGKSQP KAQNPARLSRKELENFDVYS or splice variants thereof. Amino acids 1 to 21 are a signal sequence which may be processed in the mature form. Amino acids 22 to 127 are in the extracellular domain. A known variant is N89D.

The term “CD48” refers to the naturally occurring molecule in humans identified as having (SEQ ID NO: 18) MCSRGWDSCL ALELLLLPLS LLVTSIQGHL VHMTVVSGSN VTLNISESLP ENYKQLTWFY TFDQKIVEWD SRKSKYFESK FKGRVRLDPQ SGALYISKVQ KEDNSTYIMR VLKKTGNEQE WKIKLQVLDP VPKPVIKIEK IEDMDDNCYL KLSCVIPGES VNYTWYGDKR PFPKELQNSV LETTLMPHNY SRCYTCQVSN SVSSKNGTVC LSPPCTLARS and splice variants thereof. Amino acids 1 to 26 are a signal sequence which may be processed in the mature form. Amino acids 27 to 127 are in the extracellular domain. A known variant is E102Q. CD48 is a GPI-anchored glycoprotein that is also receptor for CD2. CD48 is expressed on peripheral blood T, B and null cells, thymocytes, eosinophils, a portion of bone marrow cells and some epithelial cells.

### Sepsis-Induced Immune Dysregulation

As used herein, the term “sepsis” refers to life-threatening organ dysfunction caused by a dysregulated host response to infection. Sepsis is a condition which injures tissues or organs that arises in the presence of a persistent infection, typically bacterial, but it can also be from fungi, viruses, or parasites. Common signs and symptoms include fever, increased heart rate, and increased breathing rate accompanied with symptoms related to an infection. Septic shock refers to a subset of sepsis in which particularly profound circulatory, cellular, and metabolic abnormalities are associated with a greater risk of mortality than with sepsis alone.

Diagnosis of sepsis may be based on Sequential [Sepsis-related] Organ Failure Assessment (SOFA) score of 2 points or more (See Singer et al. The Third International Consensus Definitions for Sepsis and Septic Shock (Sepsis-3) JAMA. 2016; 315(8):801-810) or by meeting at least two systemic inflammatory response syndrome (SIRS) criteria due to a presumed infection. Systemic inflammatory response syndrome (SIRS) criteria are: abnormal body temperature (temperature>38° C. or <36° C.), heart rate (heart rate>90/min), respiratory rate or blood gas (respiratory rate>20/min or Paco2<32 mm Hg (4.3 kPa)), and white blood cell count (white blood cell count>12 000/mm3 or <4000/mm3 or >10% immature bands).

Some patients such as immune compromised individuals, e.g., over 65 years of age, taking immune suppression drugs, may not show symptoms of an infection. Severe sepsis is sepsis with sepsis-induced organ dysfunction or tissue hypoperfusion, typically manifesting as hypotension, elevated lactate, or decreased urine output. Septic shock is severe sepsis plus persistently low blood pressure despite the administration of intravenous fluids. Patients with septic shock can be clinically identified by a vasopressor, i.e. failure to maintain a mean arterial pressure of 65 mm Hg or greater and serum lactate level greater than 2 mmol/L (>18 mg/dL) in the absence of hypovolemia. Risk factors for sepsis include young age, e.g, less than 1 or 2 years of age, or old age, e.g., more than 60 or 65 years of age, a weakened immune system, e.g., from conditions such as cancer or diabetes, taking drug for immune suppression, and as the result of major trauma or burns. Sepsis is usually treated with intravenous fluids and antibiotics. If fluid replacement is not enough to maintain blood pressure, medications that raise blood pressure can be used.

Signs of organ dysfunction or failure vary depending on the organ. For lung failure one may be diagnosed with acute respiratory distress syndrome (ARDS). For the brain one may be diagnosed with encephalopathy based on symptoms including agitation, confusion, and coma. Liver failure disrupts protein synthesis resulting in progressive disruption of blood clotting and elevated serum bilirubin levels. Kidney failure typically results in low urine output, electrolyte abnormalities, or volume overload. Heart failure includes systolic and diastolic heart failure.

Table of Sequential [Sepsis-related] Organ Failure Assessment (SOFA)

| | Score | | | | |
|---|---|---|---|---|---|
| | 0 | 1 | 2 | 3 | 4 |
| Pao₂/Fio₂, mmHg | ≧400 (53.3) | <400 (53.3) | <300 (40) | <200 (26.7) with | <100 (13.3) with |
| (kPa) | | | | respiratory support | respiratory support |
| Platelets, ×10³/μL | ≧150 | <150 | <100 | <50 | <20 |
| Bilirubin, mg/dL | <1.2 (20) | 1.2-1.9 (20-32) | 2.0-5.9 (33-101) | 6.0-11.9 (102-204) | >12.0 (204) |
| (μmol/L) | | | | | |
| | MAP ≧70 mmHg | MAP <70 mmHg | Dopamine <5 or | Dopamine 5.1-15 | Dopamine >15 or |
| | | | dobutamine (any dose) | or epinephrine ≦0.1 | epinephrine >0.1 |
| | | | | or norepinephrine ≦0.1 | or norepinephrine >0.1 |
| Glasgow Coma Scale | 15 | 13-14 | 10-12 | 6-9 | <6 |
| score | | | | | |
| Creatinine, mg/dL | <1.2 (110) | 1.2-1.9 (110-170) | 2.0-3.4 (171-299) | 3.5-4.9 (300-440) | >5.0 (440) |
| (μmol/L) | | | | | |
| Urine output, mL/d | | | | <500 | <200 |

For cardiovascular function the mean arterial pressure (MAP) is evaluated after catcholamine dose at ug/kg/min for at least one hour.

In order to determine whether 2B4 or CD48 as coinhibitory molecules participate in the immunosuppressive phase that leads to mortality during sepsis experiments were performed. 2B4 (CD244, SLAMf4) is a member of the CD2 subset of immunoglobulin superfamily molecules. 2B4 has role on NK cells and can be inducibly expressed on CD4+ and CD8+ T cells. 2B4 possesses both activating and inhibitory functions, which are dependent on the level of 2B4 expression, degree of binding by its ligand CD48, and level of intracellular association with the adaptor molecule SLAM-associated protein (SAP). The role of 2B4 during a cecal ligation and puncture (CLP) animal model of sepsis were determined. 2B4 signals mediate sepsis-induced immune dysregulation and mortality believed to be via a CD4+ T cell dependent mechanism.

### Specific Binding Agents and Antibodies

This disclosure relates to methods of treating or preventing sepsis, severe sepsis, or septic shock comprising administering an effective amount of a 2B4 antibody, an CD48 variant, a CD48 antibody or specific binding agent thereof to a subject in need thereof. The subject may be diagnosed with, at risk of, or exhibiting symptoms of sepsis. In certain embodiments, the antibody binds near Lys68 and Glu70 in the extracellular domain of human 2B4. In certain embodiments, the antibody is a monoclonal antibody clone C1.7 or variant thereof.

In certain embodiments, the specific binding agent is an antibody that specifically binds human CD48. In certain embodiments, the specific binding agent specifically binds human CD48 extracellular domain. In certain embodiments, the specific binding agent is a monoclonal antibody. In certain embodiments, the specific binding agent or antibody is a humanized antibody, chimera, or fragment thereof.

Suitable specific binding agents may be prepared using methods known in the art. An exemplary 2B4 or CD48 polypeptide specific binding agent of the present disclosure is capable of binding a certain portion of the 2B4 or CD48 polypeptides, and preferably modulating the activity or function of 2B4 or CD48 polypeptides. Specific binding agents such as antibodies and antibody fragments that specifically bind 2B4 or CD48 polypeptides are within the scope of the present disclosure. The antibodies may be polyclonal including mono-specific polyclonal, monoclonal (mAbs), recombinant, chimeric, humanized such as CDR-grafted, human, single chain, catalytic, multi-specific and/or bi-specific, as well as antigen-binding fragments, variants, and/or derivatives thereof.

In certain embodiments, the disclosure contemplate 2B4 and CD48 antibodies and specific binding agents derived thereof identified by analyzing the DNA or RNA of antigen secreting cells or hydridoma of such antibodies. One can identify the six CDR regions within light and heavy chain variable regions that allow for antigen binding, i.e., “CDRs” using the following method:

CDR-L1—Starts at about residue 24. Residue before is typically a Cys. Length is typically 10 to 17 residues the residue after is a Trp. Typically Trp-Tyr-Gln, but also, Trp-Leu-Gln, Trp-Phe-Gln, Trp-Tyr-Leu.

CDR-L2—Starts about 16 residues after the end of L1. Residues before is typically Ile-Tyr, but also, Val-Tyr, Ile-Lys, Ile-Phe. Length is typically 7 residues.

CDR-L3—Starts about 33 residues after end of L2. The residue before is Cys, and the length is typically 7 to 11 residues. The residues after are typically Phe-Gly-XXX-Gly, wherein XXX is any amino acid.

CDR-H1—Starts at about residue 26 (5 after a Cys). The length is typically 9 to 12 residues. The residue after is typically a Trp. Typically Trp-Val, but also, Trp-Ile, Trp-Ala

CDR-H2—Starts about 15 residues after the end of CDR-H1. The length is typically 16 to 19 residues. The residues before are typically Leu-Glu-Trp-Ile-Gly, but a number of variations. Residues after are typically Lys/Arg-Leu/Ile/Val/Phe/Thr/Ala-Thr/Ser/Ile/Ala

CDR-H3—Starts about 33 residues after end of CDR-H2 (2 after a Cys). The length may be 3 to 25 residues. Residues before are typically Cys-XXX-XXX (such as Cys-Ala-Arg) and the residues after typically Trp-Gly-XXX-Gly.

Valiante & Trinchieri report a monoclonal antibody (mAb C1.7) that binds a 38-kD signal-transducing surface molecule (p38), subsequently identified as 2B4, expressed by lymphocyte subsets capable of cell-mediated cytotoxicity. J. Exp. Med. (1993) 178, 1397-1406. See also U.S. Pat. No. 5,688,690. Human 2B4 contains an extracellular domain, transmembrane region, and cytoplasmic domain that contains immunoreceptor tyrosine-based inhibitory motifs typically of the immunoglobulin super family. Brown et al. report 2B4 is a ligand for CD48. J Exp Med, 188 (1998), p. 2083. Mathew report that mutational analysis of the human 2B4 (CD244)/CD48 interaction and that Lys68 and Glu70 in the V domain of 2B4 are critical for CD48 binding and functional activation of NK cells. J Immunol. 2005, 175(2):1005-13.

The variable regions of the light and heavy chains of the C1.7 monoclonal antibody may be sequenced from the hybridoma cell line deposited as ATCC HB 11717. Wrammert et al. report using immunoglobulin variable regions isolated from sorted single antibody secreting cells (ASCs) to produce human monoclonal antibodies (mAbs) that bound with high affinity. Nature. 2008 May 29; 453(7195): 667-671. Smith et al. report a protocol for the production of antigen-specific human monoclonal antibodies (hmAbs) wherein ASCs are isolated from whole blood collected after vaccination and sorted by flow cytometry into single cell plates. The antibody genes of the ASCs are then amplified by RT-PCR and nested PCR, cloned into expression vectors and transfected into a human cell line.

Alternatively, one generates fully human mAbs from nonhuman variable regions using information from the human germline repertoire. Residues within and proximal to CDRs and the V_{H}/V_{L} interface are iteratively explored for substitutions to the closest human germline sequences using semi-automated computational methods. See Bernett et al., J Mol. Biology, 2010, 396(5):1474-1490. One generates fully human antibodies with substitutions compared to the parent murine sequences. Substitutions may be in the CDRs.

The engineering process to generate fully human mAbs from murine Fvs consists of five main steps: (1) design of framework-optimized VH and VL template sequences of eBioC1.7 monoclonal antibody (2) identification of the closest matching human germline sequence for the framework-optimized VH and VL, (3) screening of all possible single substitutions that increase the sequence identity of the framework-optimized sequence to the closest human germline sequence, (4) screening of VH and VL variants consisting of combinations of neutral or affinity enhancing single substitutions, and (5) screening of the highest-affinity VH and VL pairs to generate the final fully human mAb.

One defines two principal scores used to measure sequence humanness. Human identity is defined as the number of exact sequence matches between the Fv and the highest identity human germline VH, Vκ, JH, and Jκchains (the D-segment for the heavy chain is not included). The second score is the number of total “human 9-mers”, which is an exact count of 9-mer stretches in the Fv that perfectly match any one of the corresponding stretches of nine amino acids in our set of functional human germline sequences. Both human 9-mers and human identity are expressed as percentages throughout in order to enable comparison between antibody Fvs of different lengths.

Hosen et al. report CD48 as a novel molecular target for antibody therapy in multiple myeloma and the generation of anti-CD48 mAbs. Br J Haematol. 2012, 156(2):213-24. U.S. Patent Application Publication Number 20120045446 (Hosen et al.) report anti-human CD48 antibody sequences wherein the heavy chain is (SEQ ID NO: 1):

QVQLQQSGAELVRPGTSVKMSCKAAGYTFTNYWIGWVKERPGHGLEWIGDI YPGGGFTNYNENFKGKATLTADTSSSTAYMQLSSLTSEDSAIYHCARGIYYNSSPYFDS WGQGTTLT, and

wherein the light chain is (SEQ ID NO: 2)

DIVMTQFAGVDGDIVMTQSHKFMSTSVGDRVSITCKASQDVSTTVAWYQQKPG QSPKLLIYSAYRYTGVPDRFTGSGSGTDFTFTISSVQAEDLAVYYCQQHYSTPPTFGGGT KLEIK

In certain embodiment, the anti-CD48 antibody or specific binding agent of this disclosure for use in methods disclosed herein comprises the six CDRs wherein the light chain comprises (SEQ ID NO: 3) KASQDVSTTVA, (SEQ ID NO: 4) SAYRYTG, (SEQ ID NO: 5) QQHYSTPPT, and the heavy chain comprises (SEQ ID NO: 6) TFTNYWIG, (SEQ ID NO: 7) DIYPGGGFTNYNENF, (SEQ ID NO: 8) GIYYNSSPYFDS.

U.S. Patent Application Publication Number 20120076790 (Classon et al.) report an anti-CD48 antibody with a heavy chain variable region (HCVR) having the amino acid sequence of (SEQ ID NO:15) EVQLLESGGGLVHPGGSLRLSCAASGFTFGGYAMSWVRQAPGKGLEW VSLISGSGGSTYYADSVKGRFTIFRDNSKNTLYLQMISLRAEDSAVYYCAKYSNYDYFD PWGQGTLVTVSS and the CDRs of a light chain variable region (LCVR) having the amino acid sequence of (SEQ ID NO: 16) EIVLTQSPGTLSLSPGERVTLSCRASQSVSSSYLAWY QQKPGQAPRLLIYGASSRATGIPDRFSGSGSGTDFTLTISRLEPEDFAVYYCQQYGSSPRT FGQGTKVEIK.

The anti-human CD48 antibody CDRs of the heavy and light chain sequences (SEQ ID NO: 9) GFTFGGYA, (SEQ ID NO: 10) ISGSGGST, (SEQ ID NO: 11) AKYSNYDYFDP, (SEQ ID NO: 12) QSVSSSY, (SEQ ID NO: 13) GAS and (SEQ ID NO: 14) QQYGSSPRT. In certain embodiment, the anti-CD48 antibody of specific binding agent of this disclosure for use in methods disclosed herein comprises the six CDRs of SEQ ID NO: 9-14.

Once heavy or light chain sequences or polynucleotide sequences are identified which encode each chain of the full length monoclonal antibody or the Fab or Fv fragment(s) of the disclosure, host cells, either eukaryotic or prokaryotic, may be used to express the monoclonal antibody polynucleotides using recombinant techniques well known and routinely practiced in the art. Alternatively, transgenic animals may be produced wherein a polynucleotide encoding the desired specific antibody, heavy chain, light chain, and/or binding agent amino acid sequence is introduced into the genome of a recipient animal, such as, for example, a mouse, rabbit, goat, or cow, in a manner that permits expression of the polynucleotide molecules encoding a monoclonal antibody or other specific binding agent. In one aspect, the polynucleotides encoding the monoclonal antibody or other specific binding agent can be ligated to mammary-specific regulatory sequences, and the chimeric polynucleotides can be introduced into the germline of the target animal. The resulting transgenic animal then produces the desired antibody in its milk [Pollock et al., J Immunol Meth 231:147-157 (1999); Little et al., Immunol Today 8:364-370 (2000)]. In addition, plants may be used to express and produce 2B4 or CD48 or specific binding agents such as monoclonal antibodies by transfecting suitable plants with the polynucleotides encoding the monoclonal antibodies or other specific binding agents.

In another embodiment of the present disclosure, a monoclonal or polyclonal antibody or fragment thereof that is derived from other than a human species may be “humanized” or “chimerized”. Methods for humanizing non-human antibodies are well known in the art. (see U.S. Pat. Nos. 5,859,205, 5,585,089, and 5,693,762). Humanization is performed, for example, using methods described in the art [Jones et al., Nature, 321: 522-525 (1986); Riechmann et al., Nature, 332: 323-327 (1988); Verhoeyen et al., Science, 239:1534-1536 (1988)] by substituting at least a portion of, for example a rodent, complementarity-determining region (CDRs) for the corresponding regions of a human antibody. The disclosure also provides variants and derivatives of these human antibodies as discussed herein and well known in the art.

Also encompassed by the disclosure are fully human antibodies that bind 2B4 or CD48 or polypeptides, as well as, antigen-binding fragments, variants and/or derivatives thereof. Alternatively, transgenic animals (e.g., mice) that are capable of producing a repertoire of human antibodies in the absence of endogenous immunoglobulin production can be used to generate such antibodies. This can be accomplished by immunization of the animal with a 2B4 or CD48 or antigen or specific binding agents thereof where the 2B4 or CD48 or specific binding agents have an amino acid sequence that is unique to 2B4 or CD48. Such immunogens can be optionally conjugated to a carrier. See, for example, Jakobovits et al., Proc Natl Acad Sci (USA), 90: 2551-2555 (1993); Jakobovits et al., Nature 362: 255-258 (1993); Bruggermann et al., Year in Immuno, 7: 33 (1993). In one method, such transgenic animals are produced by incapacitating the endogenous loci encoding the heavy and light immunoglobulin chains therein, and inserting loci encoding human heavy and light chain proteins into the genome thereof. Partially modified animals, which are those having less than the full complement of these modifications, are then crossbred to obtain an animal having all of the desired immune system modifications. When administered an immunogen, these transgenic animals are capable of producing antibodies with human variable regions, including human (rather than e.g., murine) amino acid sequences, that are immuno-specific for the desired antigens. See PCT application Nos., PCT/US96/05928 and PCT/US93/06926. Additional methods are described in U.S. Pat. No. 5,545,807, PCT application Nos. PCT/US91/245, PCT/GB89/01207, and in EP 546073B1 and EP 546073A1. Human antibodies may also be produced by the expression of recombinant DNA in host cells or by expression in hybridoma cells as described herein.

Transgenesis is achieved in a number of different ways. See, for example, Bruggeman et al., Immunol Today 17:391-7 (1996). In one approach, a minilocus is constructed such that gene segments in a germline configuration are brought artificially close to each other. Due to size limitations (i.e., having generally less than 30 kb), the resulting minilocus will contain a limited number of differing gene segments, but is still capable of producing a large repertoire of antibodies. Miniloci containing only human DNA sequences, including promoters and enhancers are fully functional in the transgenic mouse.

When larger number of gene segments are desired in the transgenic animal, yeast artificial chromosomes (YACs) or bacterial artificial chromosomes (BACs) may be utilized. YACs can range from several hundred kilobases to 1 Mb and are introduced into the mouse (or other appropriate animal) genome via microinjection directly into an egg or via transfer of the YAC into embryonic stem (ES)-cell lines. In general, YACs are transferred into ES cells by lipofection of the purified DNA, or yeast spheroplast fusion wherein the purified DNA is carried in micelles and fusion is carried out in manner similar to hybridoma fusion protocols. Selection of desired ES cells following DNA transfer is accomplished by including on the YAC any of the selectable markers known in the art.

As another alternative, bacteriophage P1 vectors are used which are amplified in a bacterial E. coli host. While these vectors generally carry less inserted DNA than a YAC, the clones are readily grown in high enough yield to permit direct microinjection into a mouse egg. Use of a cocktail of different P1 vectors has been shown to lead to high levels of homologous recombination.

Human antibodies can also be produced by exposing human splenocytes (B or T cells) to an antigen in vitro, then reconstituting the exposed cells in an immunocompromised mouse, e.g. SCID or nod/SCID. See Brams et al., J Immunol, 160: 2051-2058 [1998]; Carballido et al., Nat Med, 6: 103-106 [2000]. In one approach, engraftment of human fetal tissue into SCID mice (SCID-hu) results in long-term hematopoiesis and human T-cell development [McCune et al., Science 241:1532-1639 (1988); Ifversen et al., Sem Immunol 8:243-248 (1996)]. Any humoral immune response in these chimeric mice is completely dependent on co-development of T-cells in the animals [Martensson et al., Immunol 83:1271-179 (1994)]. In an alternative approach, human peripheral blood lymphocytes are transplanted intraperitoneally (or otherwise) into SCID mice [Mosier et al., Nature 335:256-259 (1988)]. When the transplanted cells are treated with either a priming agent, such as Staphylococcal Enterotoxin A (SEA) [Martensson et al., Immunol 84: 224-230 (1995)], or anti-human CD40 monoclonal antibodies [Murphy et al., Blood 86:1946-1953 (1995)], higher levels of B cell production are detected.

Alternatively, an entirely synthetic human heavy chain repertoire is created from unrearranged V gene segments by assembling each human VH segment with D segments of random nucleotides together with a human J segment [Hoogenboom et al., J Mol Biol 227:381-388 (1992)]. Likewise, a light chain repertoire is constructed by combining each human V segment with a J segment [Griffiths et al., EMBO J. 13:3245-3260 (1994)]. Nucleotides encoding the complete antibody (i.e., both heavy and light chains) are linked as a single chain F_{V} fragment and this polynucleotide is ligated to a nucleotide encoding a filamentous phage minor coat protein. When this fusion protein is expressed on the surface of the phage, a polynucleotide encoding a specific antibody is identified by selection using an immobilized antigen.

In still another approach, antibody fragments are assembled as two Fab fragments by fusion of one chain to a phage protein and secretion of the other into bacterial periplasm [Hoogenboom et al., Nucl Acids Res 19:4133-4137 [1991]; Barbas et al., Proc Natl Acad Sci (USA) 88:7978-7982 (1991)].

Large-scale production of chimeric, humanized, CDR-grafted, and fully human antibodies, or specific binding agents, are typically produced by recombinant methods. Polynucleotide molecule(s) encoding the heavy and light chains of each antibody or specific binding agents, can be introduced into host cells and expressed using materials and procedures described herein. In a preferred embodiment, the antibodies are produced in mammalian host cells, such as CHO cells.

The specific binding agents of the present disclosure, such as the antibodies, antibody fragments, and antibody derivatives of the disclosure can further comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutant of a naturally occurring constant region.

In one embodiment, the specific binding agents of the present disclosure, such as the antibodies, antibody fragments, and antibody derivatives of the disclosure comprise an IgG.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

The specific binding agents of the present disclosure, such as the antibodies, antibody fragments, and antibody derivatives of the disclosure may comprise the IgG1 heavy chain constant domain or a fragment of the IgG1 heavy chain domain. The antibodies, antibody fragments, and antibody derivatives of the disclosure may further comprise the constant light chain kappa or lambda domains or a fragment of these. Light chain constant regions and polynucleotides encoding them are provided herein below. In another embodiment, the antibodies, antibody fragments, and antibody derivatives of the disclosure further comprise a heavy chain constant domain, or a fragment thereof, such as the IgG2 heavy chain constant region.

### Therapeutic Methods

Various delivery systems are known and can be used to administer the therapeutic or prophylactic compositions, e.g., encapsulation in liposomes, microparticles, microcapsules, recombinant cells capable of expressing the antibody or fusion protein, receptor-mediated endocytosis (see, e.g., Wu and Wu, 1987, J. Biol. Chem. 262:4429-4432), construction of a nucleic acid as part of a retroviral or other vector, etc.

Methods of administering antibodies and specific binding agents include, but are not limited to, parenteral administration (e.g., intradermal, intramuscular, intraperitoneal, intravenous and subcutaneous), epidural, and mucosal (e.g., intranasal and oral routes). In a specific embodiment, the antibodies or fusion proteins are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,20; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In a specific embodiment, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In some embodiments, the antibodies or specific binding agents are formulated in liposomes for targeted delivery of the antibodies or fusion proteins. Liposomes are vesicles comprised of concentrically ordered phospholipid bilayers which encapsulate an aqueous phase. Liposomes typically comprise various types of lipids, phospholipids, and/or surfactants. The components of liposomes are arranged in a bilayer configuration, similar to the lipid arrangement of biological membranes. Liposomes are particularly preferred delivery vehicles due, in part, to their biocompatibility, low immunogenicity, and low toxicity. Methods for preparation of liposomes are known in the art and are encompassed within the invention, see, e.g., Epstein et al., 1985, Proc. Natl. Acad. Sci. USA, 82: 3688; Hwang et al., 1980 Proc. Natl. Acad. Sci. USA, 77: 4030-4; U.S. Pat. Nos. 4,485,045 and 4,544,545.

Methods of preparing liposomes with a prolonged serum half-life, i.e., enhanced circulation time, such as those disclosed in U.S. Pat. No. 5,013,556 can be used to make liposomes-antibody compositions. Preferred liposomes are not rapidly cleared from circulation, i.e., are not taken up into the mononuclear phagocyte system (MPS). The invention encompasses sterically stabilized liposomes which are prepared using common methods known to one skilled in the art. Although not intending to be bound by a particular mechanism of action, sterically stabilized liposomes contain lipid components with bulky and highly flexible hydrophilic moieties, which reduces the unwanted reaction of liposomes with serum proteins, reduces opsonization with serum components and reduces recognition by MPS. Sterically stabilized liposomes are preferably prepared using polyethylene glycol. For preparation of liposomes and sterically stabilized liposome, see, e.g., Bendas et al., 2001 BioDrugs, 15(4): 215-224; Allen et al., 1987 FEBS Lett. 223: 42-6; Klibanov et al., 1990 FEBS Lett., 268: 235-7; Blum et al., 1990, Biochim. Biophys. Acta., 1029: 91-7; Torchilin et al., 1996, J. Liposome Res. 6: 99-116; Litzinger et al., 1994, Biochim. Biophys. Acta, 1190: 99-107; Maruyama et al., 1991, Chem. Pharm. Bull., 39: 1620-2; Klibanov et al., 1991, Biochim Biophys Acta, 1062; 142-8; Allen et al., 1994, Adv. Drug Deliv. Rev, 13: 285-309. The invention also encompasses liposomes that are adapted for specific organ targeting, see, e.g., U.S. Pat. No. 4,544,545, or specific cell targeting, see, e.g., U.S. Patent Application Publication No. 2005/0074403. Particularly useful liposomes for use in the disclosed compositions and methods can be generated by reverse phase evaporation method with a lipid composition comprising phosphatidylcholine, cholesterol, and PEG derivatized phosphatidylethanolamine (PEG-PE). Liposomes are extruded through filters of defined pore size to yield liposomes with the desired diameter. In some embodiments, a fragment of an antibody, e.g., F(ab′), may be conjugated to the liposomes using previously described methods, see, e.g., Martin et al., 1982, J. Biol. Chem. 257: 286-288.

The antibodies or specific binding agents may also be formulated as immunoliposomes. Immunoliposomes refer to a liposomal composition, wherein an antibody or a fragment thereof is linked, covalently or non-covalently to the liposomal surface. The chemistry of linking an antibody to the liposomal surface is known in the art and encompassed within the invention, see, e.g., U.S. Pat. No. 6,787,153; Allen et al., 1995, Stealth Liposomes, Boca Rotan: CRC Press, 233-44; Hansen et al., 1995, Biochim. Biophys. Acta, 1239: 133-144. In most preferred embodiments, immunoliposomes for use in the disclosed methods and compositions are further sterically stabilized. Preferably, the antibodies or specific binding agents are linked covalently or non-covalently to a hydrophobic anchor, which is stably rooted in the lipid bilayer of the liposome. Examples of hydrophobic anchors include, but are not limited to, phospholipids, e.g., phosoatidylethanolamine (PE), phosphatidylinositol (PI). To achieve a covalent linkage between an antibody and a hydrophobic anchor, any of the known biochemical strategies in the art may be used, see, e.g., J. Thomas August, ed., 1997, Gene Therapy: Advances in Pharmacology, Volume 40, Academic Press, San Diego, Calif., p. 399-435. For example, a functional group on an antibody molecule may react with an active group on a liposome associated hydrophobic anchor, e.g., an amino group of a lysine side chain on an antibody may be coupled to liposome associated N-glutaryl-phosphatidylethanolamine activated with water-soluble carbodiimide; or a thiol group of a reduced antibody can be coupled to liposomes via thiol reactive anchors, such as pyridylthiopropionylphosphatidylethanolamine. See, e.g., Dietrich et al., 1996, Biochemistry, 35: 1100-1105; Loughrey et al., 1987, Biochim. Biophys. Acta, 901: 157-160; Martin et al., 1982, J. Biol. Chem. 257: 286-288; Martin et al., 1981, Biochemistry, 20: 4429-38. Although not intending to be bound by a particular mechanism of action, immunoliposomal formulations including an antibody or fusion protein are particularly effective as therapeutic agents, since they deliver the antibody or fusion protein to the cytoplasm of the target cell, i.e., the cell comprising the receptor to which the antibody or fusion protein binds. The immunoliposomes preferably have an increased half-life in blood, specifically target cells, and can be internalized into the cytoplasm of the target cells thereby avoiding loss of the therapeutic agent or degradation by the endolysosomal pathway.

The immunoliposomal compositions include one or more vesicle forming lipids, an antibody or a fragment or derivative thereof or a fusion protein, and, optionally, a hydrophilic polymer. A vesicle forming lipid is preferably a lipid with two hydrocarbon chains, such as acyl chains and a polar head group. Examples of vesicle forming lipids include phospholipids, e.g., phosphatidylcholine, phosphatidylethanolamine, phosphatidic acid, phosphatidylinositol, sphingomyelin, and glycolipids, e.g., cerebrosides, gangliosides. Additional lipids useful in the formulations are known to one skilled in the art and encompassed within the invention. In some embodiments, the immunoliposomal compositions further comprise a hydrophilic polymer, e.g., polyethylene glycol, and ganglioside GM1, which increases the serum half-life of the liposome. Methods of conjugating hydrophilic polymers to liposomes are well known in the art and encompassed within the invention. For a review of immunoliposomes and methods of preparing them, see, e.g., U.S. Patent Application Publication No. 2003/0044407; PCT International Publication No. WO 97/38731, Vingerhoeads et al., 1994, Immunomethods, 4: 259-72; Maruyama, 2000, Biol. Pharm. Bull. 23(7): 791-799; Abra et al., 2002, Journal of Liposome Research, 12(1&2): 1-3; Park, 2002, Bioscience Reports, 22(2): 267-281; Bendas et al., 2001 BioDrugs, 14(4): 215-224, J. Thomas August, ed., 1997, Gene Therapy: Advances in Pharmacology, Volume 40, Academic Press, San Diego, Calif., p. 399-435.

The antibodies and specific binding agents can be packaged in a hermetically sealed container, such as an ampoule or sachette, indicating the quantity of antibody. In one embodiment, the antibodies are supplied as a dry sterilized lyophilized powder or water free concentrate in a hermetically sealed container and can be reconstituted, e.g., with water or saline to the appropriate concentration for administration to a subject. Preferably, the antibodies or fusion proteins are supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized antibodies or specific binding agents should be stored at between 2 and 8 degrees C. in their original container and the antibodies should be administered within 12 hours, preferably within 6 hours, within 5 hours, within 3 hours, or within 1 hour after being reconstituted. In an alternative embodiment, antibodies or fusion proteins are supplied in liquid form in a hermetically sealed container indicating the quantity and concentration of the antibody, fusion protein, or conjugated molecule. Preferably, the liquid form of the antibodies or fusion proteins are supplied in a hermetically sealed container at least 1 mg/ml, more preferably at least 2.5 mg/ml, at least 5 mg/ml, at least 8 mg/ml, at least 10 mg/ml, at least 15 mg/kg, at least 25 mg/ml, at least 50 mg/ml, at least 100 mg/ml, at least 150 mg/ml, at least 200 mg/ml of the antibodies of fusion proteins.

The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. For antibodies and fusion proteins, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies or specific binding agents, or fusion proteins may be reduced by enhancing uptake and tissue penetration of the antibodies or fusion proteins by modifications such as, for example, lipidation.

In certain embodiments, the therapeutic or prophylactic composition is a nucleic acid encoding a 2B4 or CD48 antibody or specific binding agent. The nucleic acid can be administered in vivo to promote expression of its encoded antibody or fragment, by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (See e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

The compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, the disclosed compositions include a prophylactically or therapeutically effective amount of antibody or fusion protein and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

One embodiment provides a pharmaceutical pack or kit comprising one or more containers filled with antibody or antigen binding fragment. Additionally, one or more other prophylactic or therapeutic agents useful for the treatment of a disease can also be included in the pharmaceutical pack or kit. One embodiment provides a pharmaceutical pack or kit including one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more antibodies or antigen binding fragments. In another embodiment, a kit further comprises one or more other prophylactic or therapeutic agents useful for the treatment of sepsis, in one or more containers.

### Combination Therapies

In certain embodiments, the antibodies or specific binding agents disclosed herein are administered in combination with an antibiotic, antiviral, or other anti-infections agent or anti-inflammatory agent.

Contemplated antibiotics include selected from the group comprising of sulfonamides, diaminopyrimidines, quinolones, Beta-lactam antibiotics, cephalosporins, tetracyclines, notribenzene derivatives, aminoglycosides, macrolide antibiotics, polypeptide antibiotics, nitrofuran derivatives, nitroimidazoles, nicotinin acid derivatives, polyene antibiotics, imidazole derivatives or glycopeptide, cyclic lipopeptides, glycylcyclines and oxazolidinones. In further embodiments, these antibiotics include but are not limited to sulphadiazine, sulfones-[dapsone (DDS) and paraaminosalicyclic (PAS)], sulfanilamide, sulfamethizole, sulfamethoxazole, sulfapyridine, trimethoprim, pyrimethamine, nalidixic acids, norfloxacin, ciproflaxin, cinoxacin, enoxacin, gatifloxacin, gemifloxacin, grepafloxacin, levofloxacin, 1Lomefloxacin, moxifloxacin, ofloxacin, pefloxacin, sparfloxacin, trovafloxacin, penicillins (amoxicillin, ampicillin, azlocillin, carbenicillin, cloxacillin, cicloxacillin, flucloxacillin, hetacillin, oxacillin, mezlocillin, penicillin G, penicillin V, piperacillin), cephalosporins (cefacetrile, cefadroxil, cefalexin, cefaloglycin, cefalonium, cefaloridin, cefalotin, cefapirin, cefatrizine, cefazaflur, cefazedone, cefazolin, cefradine, cefroxadine, ceftezole, cefaclor, cefonicid, ceforanide, cefprozil, cefuroxime, cefuzonam, cefmetazole, cefoteta, cefoxitin, cefcapene, cefdaloxime, cefdinir, cefditoren, cefetamet, cefixime, cefmenoxime, cefodizime, cefoperazone, cefotaxime, cefotiam, cefpimizole, cefpiramide, cefpodoxime, cefteram, ceftibuten, ceftiofur, ceftiolen, ceftizoxime, ceftriaxone, cefoperazone, ceftazidime, cefepime), moxolactam, carbapenems (imipenem, ertapenem, meropenem) monobactams (aztreonam), oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, framycetin, kanamycin, neomycin, netilmicin, streptomycin, tobramycin, azithromycin, clarithromycin, dirithromycin, erythromycin, roxithromycin, telithromycin, polymyxin-B, xolistin, bacitracin, tyrothricin, notrifurantoin, furazolidone, metronidazole, tinidazole, isoniazid, pyrazinamide, ethionamide, nystatin, amphotericin-B, hamycin, miconazole, clotrimazole, ketoconazole, fluconazole, rifampacin, lincomycin, clindamycin, spectinomycin, chloramphenicol, clindamycin, colistin, fosfomycin, loracarbef, metronidazole, nitrofurantoin, polymyxin B, polymyxin B sulfate, procain, spectinomycin, tinidazole, trimethoprim, ramoplanin, teicoplanin, vancomycin, trimethoprim, sulfamethoxazole, and/or nitrofurantoin.

Contemplated antiviral agent(s) include abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate, tipranavir, trifluridine, trizivir, tromantadine, truvada, valaciclovir, valganciclovir, vicriviroc, vidarabine, viramidine, zalcitabine, zanamivir, or zidovudine, and combinations thereof.

In certain embodiments, 2B4 and CD48 antibodies or specific binding agents may be administered in combination with other active agents. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

Specific examples of compounds that can be administered with the compounds include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methyl salicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

### Examples

### 2B4 Expression was Increased on CD4+ and CD8+ T Cells Following CLP

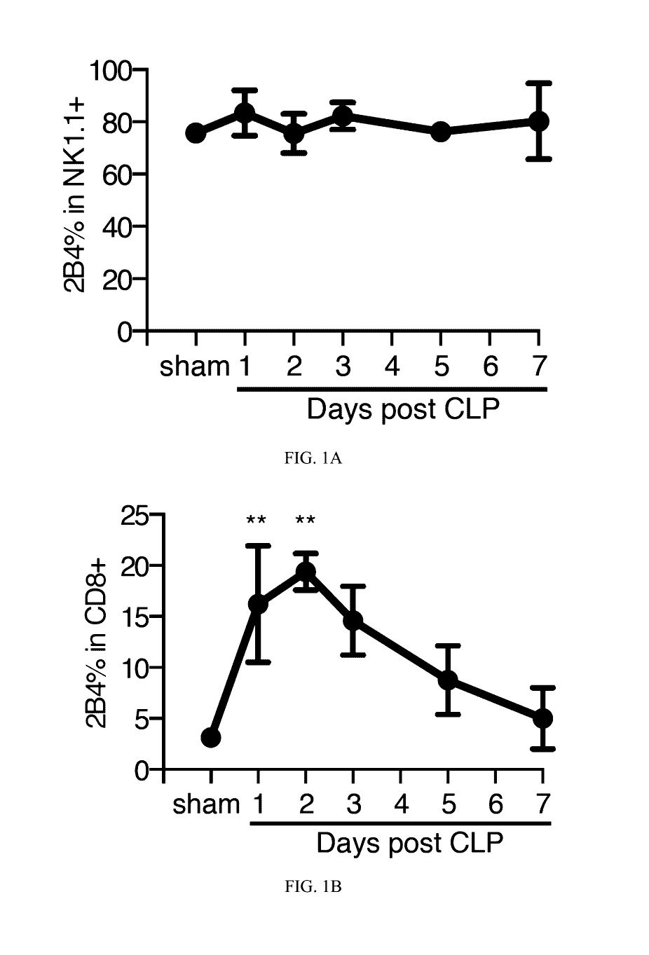

To interrogate the role of 2B4 in mediating immune dysregulation during sepsis, wild-type (WT) mice were subjected to polymicrobial sepsis via cecal ligation and puncture (CLP) and expression of 2B4 was assessed. Compared to cells isolated from animals undergoing sham surgery, NK cells expressed high level of 2B4 and did not change their expression during sepsis (FIG. 1A). Intriguingly, 2B4 expression on both CD4+ and CD8+ T cells was increased at 24 hours post-CLP (2.1% to 12.9% on CD4+s and 3.15 to 16.21% on CD8+s). The elevated expression of 2B4 on T cells was maintained for 3 days post-CLP and declined by 4 days after surgery (FIG. 1B, 1C). These data suggested that 2B4 signals might play some role in dysregulated T cell responses during sepsis.

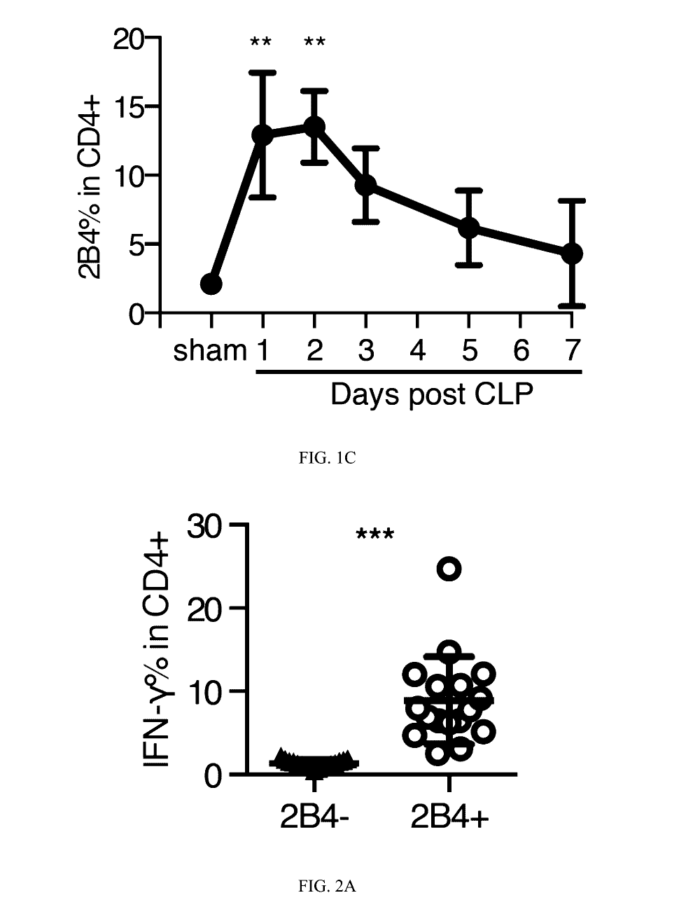

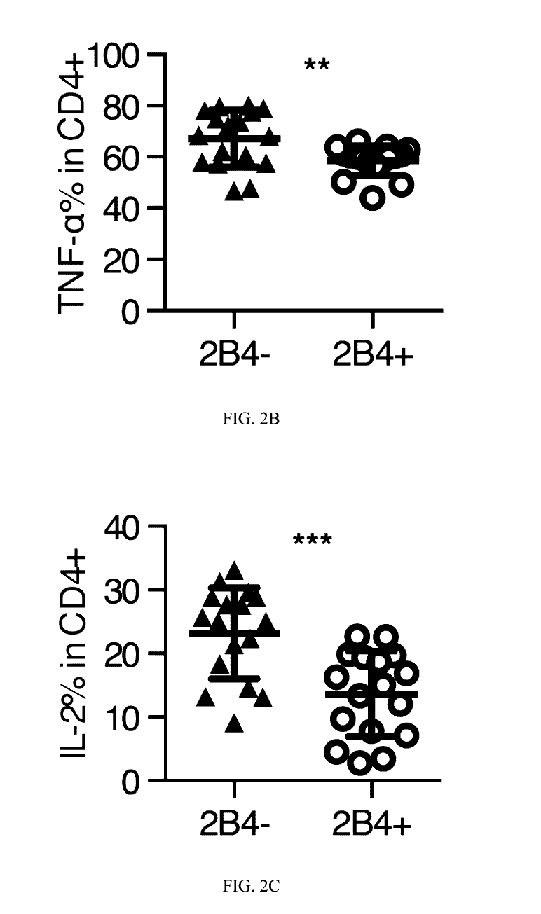

To test the effector function of 2B4-expressing T cells during sepsis, splenocytes were harvested and stimulated with PMA and Ionomycin for intracellular cytokine staining. 2B4-expressing CD4+ and CD8+ T cell populations contained higher percentages of IFN-g secreting cells relative to the 2B4− population. In contrast, 2B4-expressing populations contained lower frequencies of TNF and IL-2 secreting cells (FIG. 2). These data suggested that 2B4 expression might contribute to dysregulated T cell responses during sepsis.

2B4-Deficient Mice were Protected from Sepsis-Induced Mortality Following CLP

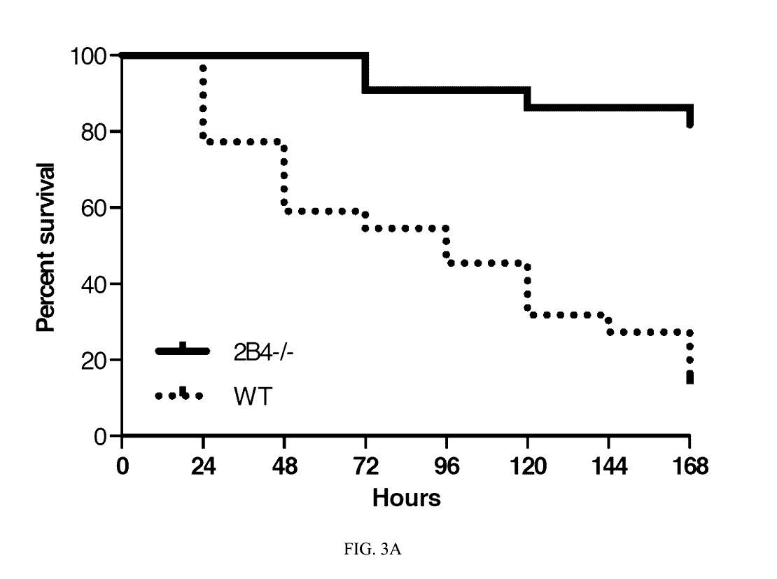

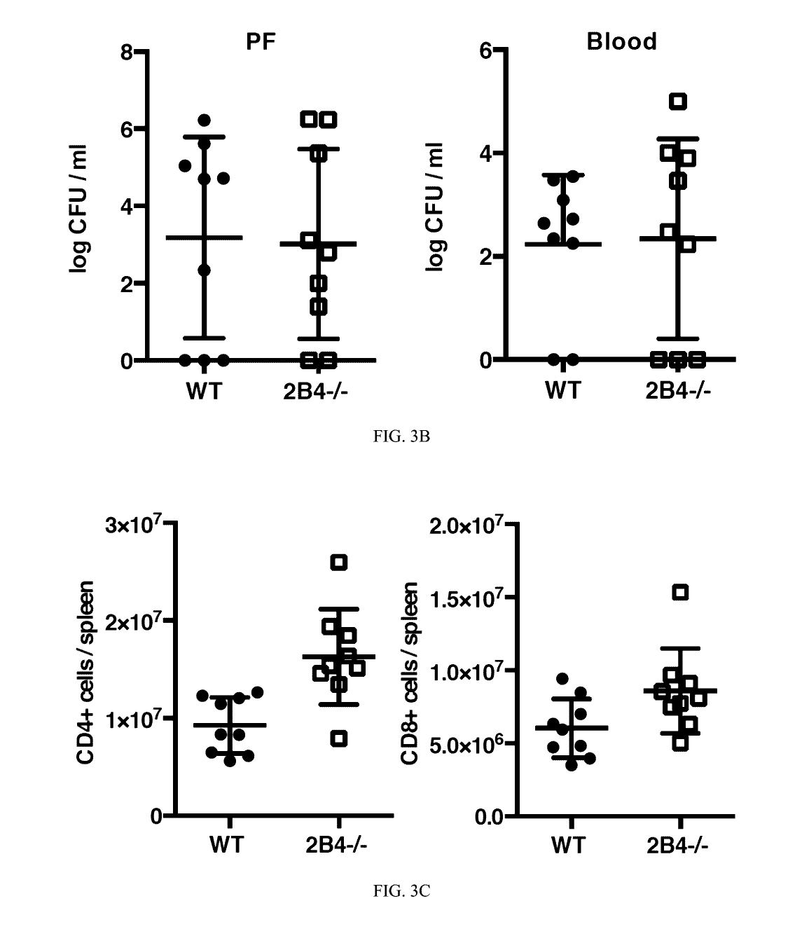

To test the effect of disrupting 2B4 signaling during sepsis, CLP was performed on eight to twelve week old 2B4−/− mice or WT mice and animals were monitored for 7 days survival. Strikingly, 2B4−/− mice were significantly protected from death during sepsis as compared to WT controls (82% survival compared to 13%, FIG. 3A). While no difference was found in the bacteria load of peritoneal fluid and plasma at 24 hours post CLP, 2B4−/− mice did possess increased numbers of CD4 and CD8 T cells (FIG. 3B, 3C). These data indicated that the survival benefit of 2B4−/− mice may result from preserved adaptive immune system (i.e. reduced lymphocyte apoptosis during sepsis).

Effector T Cells Isolated from Septic 2B4−/− Animals Possess Increased Functionality Relative to WT Controls

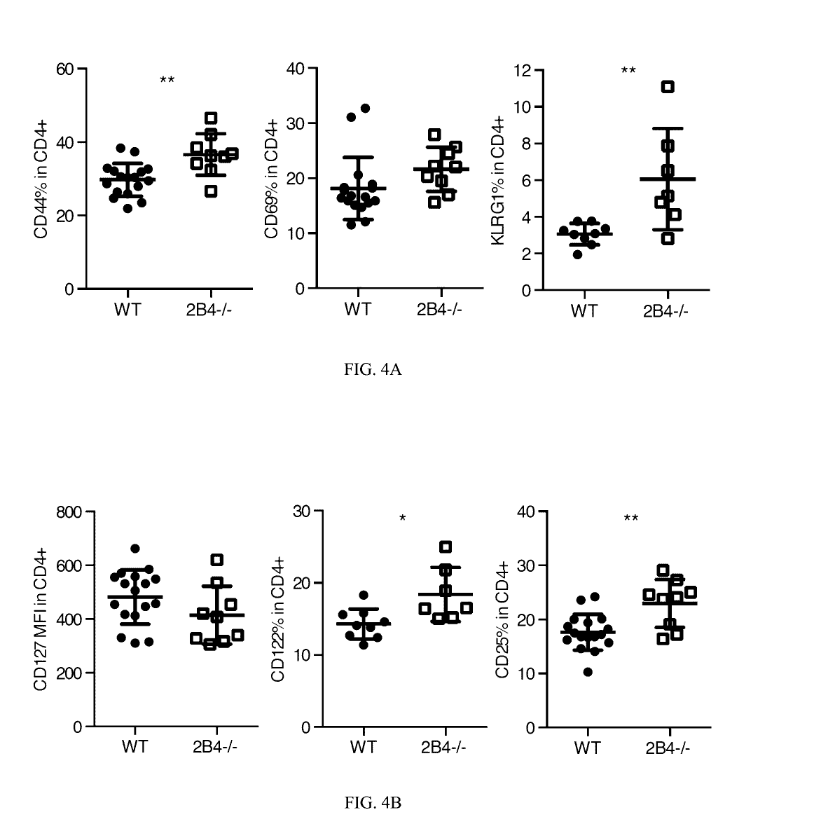

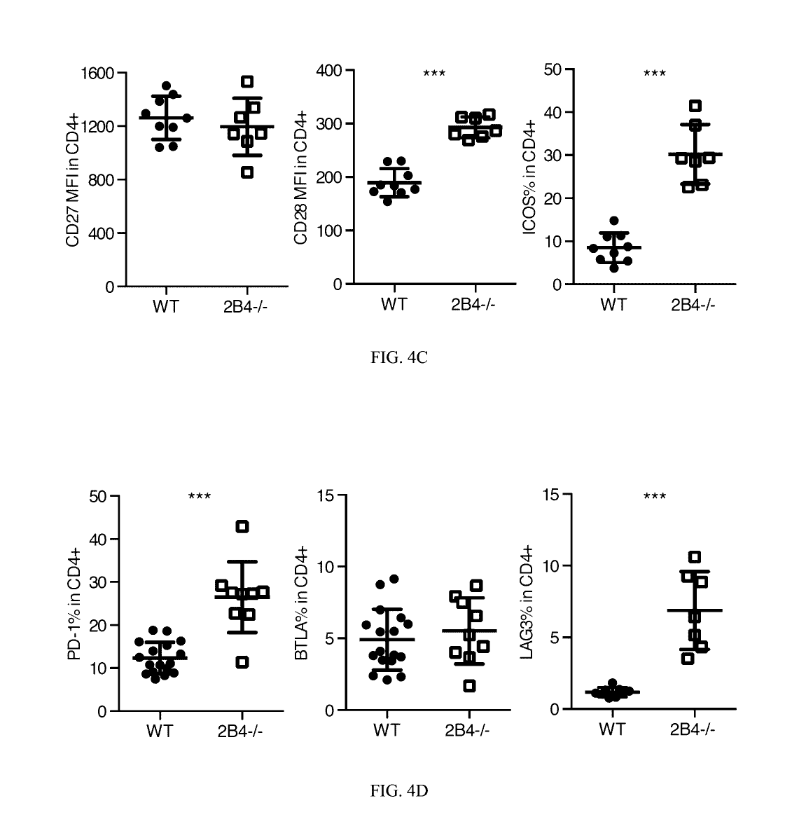

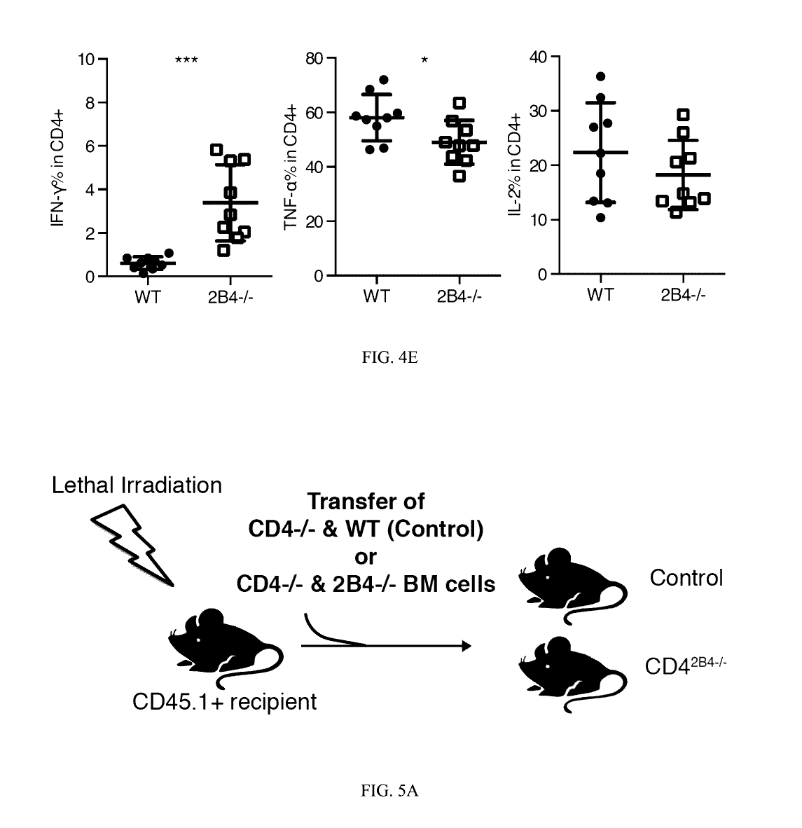

To further investigate T cell function in 2B4−/− mice, CLP was performed on WT or 2B4−/− mice and animals were sacrificed at 24 hours post CLP. Splenocytes in both groups were harvested and stained for surface markers to dissect CD4+ and CD8+ T cell phenotypes. 2B4-deficient CD4+ T cells exhibited higher CD44 and KLRG1 expression than WT CD4 T cells during sepsis (FIG. 4A). 2B4−/− CD4+ T cells also expressed higher IL-2 receptor alpha and beta chain than WT CD4+ T cells (FIG. 4B). For costimulatory receptors, higher expression of CD28 and ICOS were found in 2B4−/− mice (FIG. 4C). Moreover, 2B4−/− CD4+ T cells also had increased PD-1 and LAG-3 expression (FIG. 4D).

To determine the functionality of these cells, T cells were stimulated with PMA and ionomycin for intracellular cytokine staining. 2B4−/− CD4+ T cells showed higher percentage of IFN-g secreting cells but lower frequency of IL-2 secreting cells as compared to WT CD4+ T cells during sepsis (FIG. 4E). Taken together, this data indicates that following sepsis induction, 2B4−/− CD4+ T cells exhibited more effector phenotypes and have higher functionality, which might be a possible explanation for increased survival in 2B4-deficient mice. Similar results were obtained for 2B4−/− CD8+ T cells during sepsis.

### Loss of 2B4 Specifically on CD4+ T Cells Provides the Survival Benefit During Sepsis

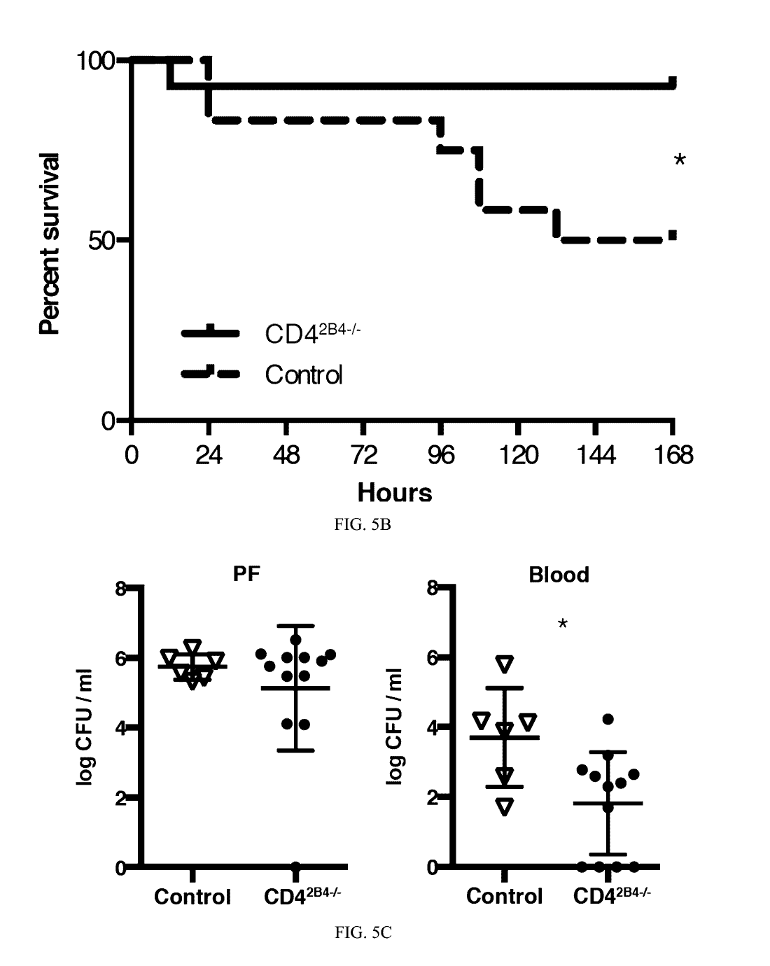

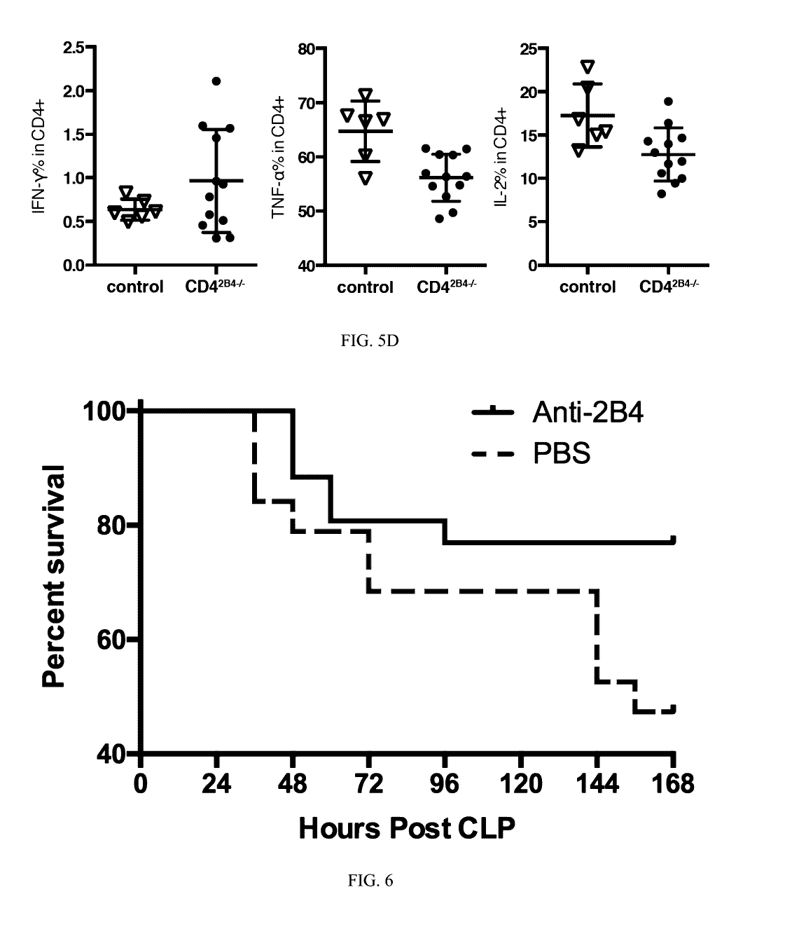

To dissect which immune population contributes to the survival benefit observed in 2B4−/− mice, bone marrow chimeric mice were generated in which 2B4 is knocked out only in the CD4 T cell compartment (FIG. 5A). The chimeras were generated by a bone marrow transplant of 80% of CD4−/−Thy1.2+ (2B4-intact) bone marrow and 20% of 2B4−/−Thy1.1+ bone marrow (or 20% WT bone marrow as control). After 10 weeks of reconstitution, all the hematopoietic lineages except CD4+ T cells were reconstituted from the CD4−/− bone marrow, which is 2B4 intact; however, CD4+ T cells in the chimera mice can only develop from the 2B4−/− bone marrow and will expand to “fill up” the CD4+ T cell compartment. The chimera mice were bled and these phenotypes were confirmed. All the CD4+ T cells were Thy1.1+ (2B4−/−), and the majority of CD8+ T cells were Thy1.2+ (2B4 intact). Hereafter we will refer to these mice as CD42B4−/− chimeric mice. CD8-specific 2B4 knockout chimeras (CD82B4−/−) were generated in a similar manner. In this way, animals in which 2B4 is deficient specifically within either the CD4+ or CD8+ T cell compartment were generated and survival following CLP was determined relative to WT chimera controls.

Surprisingly, the CD42B4−/− chimeric mice exhibited significantly improved survival following CLP as compared to CD42B4+/+ control chimeras (93% survival compared to 50%, FIG. 5B). In contrast, CD8-specific 2B4 knockout chimeras (CD82B4−/−) did not exhibit significantly altered survival relative to CD8 2B4+/+ controls. Bacteria loads were measured in peritoneal fluid and plasma at 24 hours post CLP and lower bacteremia was found in CD42B4−/− chimera relative to CD42B4+/+ control chimeras. To interrogate functional differences in CD4+ T cells between CD42B4−/− and CD42B4+/+ chimeric animals, intracellular cytokine staining was performed, and it was found that CD42B4−/− mice exhibited increased percentages of IFN-g secreting CD4+ T cells relative to CD42B4+/+ control chimeras. These data thus strongly suggest that expression of inhibitory 2B4 molecules on CD4+, but not CD8+, T cells contribute to septic mortality.

### Pharmacologic Inhibition of 2B4 Results in a Strong Trend Toward Increased Survival Following Sepsis

In order to determine whether 2B4 could be pharmacologically targeted to improve mortality following sepsis, 2B4 signaling was blocked in septic animals with a monoclonal antibody to 2B4 (clone 2B4). Mice were subjected to CLP and received either PBS (n=19) or 500 ug of the anti-2B4 mAb (n=26) on days 0 and 2 post-CLP. Results indicated a strong trend (p=0.053) toward increased survival in the animals treated with anti-2B4 relative to PBS treated controls (FIG. 6).

2B4 is Increased on CD4+ T Cells Isolated from Human Septic Patients Relative to Healthy Controls

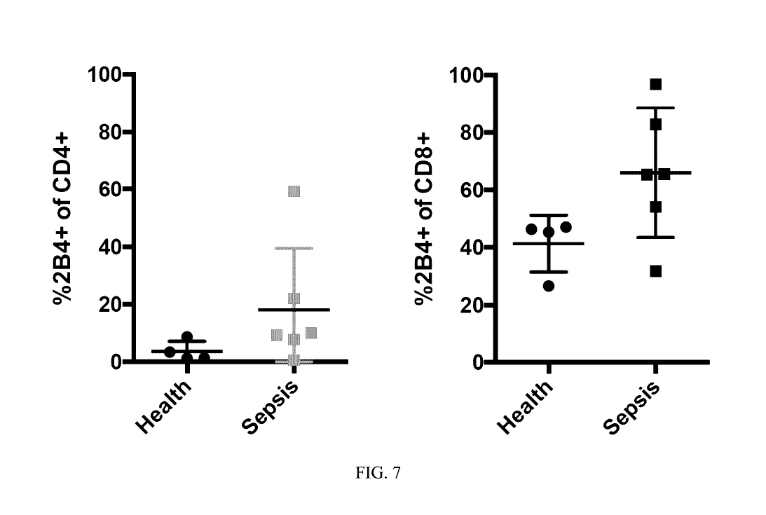

To assess the expression of 2B4 on CD4+ T cells in human septic patients, patients were enrolled in the intensive care units of Grady Memorial Hospital in an IRB-approved protocol. PBMC were collected from patients with clinically-defined sepsis in order to interrogate the expression of 2B4 on distinct immune cell types at 24 hours following sepsis onset. Preliminary analysis of 6 septic patients and 5 healthy controls reveal a strong trend towards 2B4 upregulation in both the CD4+ and CD8+ T cell compartments during sepsis (FIG. 7). Importantly, these data support the idea that 2B4 is a relevant pathway that could be therapeutically targeted during sepsis in human patients in order to improve outcomes.

1. A method of treating sepsis comprising administering an effective amount of a 2B4 specific binding agent to a subject in need thereof.

2. The method of claim 1, wherein the specific binding agent is an antibody that specifically binds 2B4.

3. The method of claim 2, wherein the antibody binds the extracellular domain of human 2B4.

4. The method of claim 3, wherein the antibody is a monoclonal antibody clone C1.7 or variant thereof.

5. The method of claim 4, wherein the specific bind agent is an antibody is a humanized antibody, chimera, or fragment thereof.

6. A method of treating sepsis comprising administering an effective amount of a CD48 variant or CD48 specific binding agent to a subject in need thereof.

7. The method of claim 6, wherein the specific binding agent is an antibody that specifically binds human CD48.

8. The method of claim 6, wherein the specific binding agent specifically binds human CD48 extracellular domain

9. The method of claim 8, wherein the antibody is a monoclonal antibody clone BJ40 or variant thereof.

10. The method of claim 8, wherein the antibody is a humanized antibody, chimera, or fragment thereof.

11. The method of claim 6, wherein the specific binding agent is an antibody having CDRs of SEQ ID NO: 3-8.

12. The method of claim 6, wherein the specific binding agent is an antibody having CDRs of SEQ ID NO: 9-14.


SEQUENCE LISTING (18 sequences)

SEQ ID NO: 1 - protein, 118 aa, Artificial sequence
  Synthetic
QVQLQQSGAELVRPGTSVKMSCKAAGYTFTNYWIGWVKERPGHGLEWIGDIYPGGGFTNYNENFKGKATLTADTSSSTAYMQLSSLTSEDSAIYHCARGIYYNSSPYFDSWGQGTTLT

SEQ ID NO: 2 - protein, 118 aa, Artificial sequence
  Synthetic
DIVMTQFAGVDGDIVMTQSHKFMSTSVGDRVSITCKASQDVSTTVAWYQQKPGQSPKLLIYSAYRYTGVPDRFTGSGSGTDFTFTISSVQAEDLAVYYCQQHYSTPPTFGGGTKLEIK

SEQ ID NO: 3 - protein, 11 aa, Artificial sequence
  Synthetic
KASQDVSTTVA

SEQ ID NO: 4 - protein, 7 aa, Artificial sequence
  Synthetic
SAYRYTG

SEQ ID NO: 5 - protein, 9 aa, Artificial sequence
  Synthetic
QQHYSTPPT

SEQ ID NO: 6 - protein, 8 aa, Artificial sequence
  Synthetic
TFTNYWIG

SEQ ID NO: 7 - protein, 15 aa, Artificial sequence
  Synthetic
DIYPGGGFTNYNENF

SEQ ID NO: 8 - protein, 12 aa, Artificial sequence
  Synthetic
GIYYNSSPYFDS

SEQ ID NO: 9 - protein, 8 aa, Artificial sequence
  Synthetic
GFTFGGYA

SEQ ID NO: 10 - protein, 8 aa, Artificial sequence
  Synthetic
ISGSGGST

SEQ ID NO: 11 - protein, 11 aa, Artificial sequence
  Synthetic
AKYSNYDYFDP

SEQ ID NO: 12 - protein, 7 aa, Artificial sequence
  Synthetic
QSVSSSY

SEQ ID NO: 13 - protein, 3 aa, Artificial sequence
  Synthetic
GAS

SEQ ID NO: 14 - protein, 9 aa, Artificial sequence
  Synthetic
QQYGSSPRT

SEQ ID NO: 15 - protein, 118 aa, Artificial sequence
  Synthetic
EVQLLESGGGLVHPGGSLRLSCAASGFTFGGYAMSWVRQAPGKGLEWVSLISGSGGSTYYADSVKGRFTIFRDNSKNTLYLQMISLRAEDSAVYYCAKYSNYDYFDPWGQGTLVTVSS

SEQ ID NO: 16 - protein, 108 aa, Artificial sequence
  Synthetic
EIVLTQSPGTLSLSPGERVTLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 17 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 18 - protein, 220 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS